(19) United States
(12) Patent Application Publication
(10) Pub. No.: US 2002/0091074 A1
(43) Pub. Date: Jul. 11, 2002

(54) Medical compositions, dressings and methods for treating microbial infections of skin lesions

(76) Inventors: Richard E. Wooley, Athens, GA (US); Branson W. Ritchie, Athens, GA (US)

Correspondence Address: Womble Carlyle Sandridge & Rice, PLLC, P.O. Box 7037, Atlanta, GA, 30357-0037, US

(21) Appl. No.: 09/955,657

(22) Filed: Sep. 18, 2001

Related U.S. Application Data
(60) Provisional application No. 60/234,375, filed on Sep. 20, 2000.

(51) Int. Cl. A61K 31/00; C12Q 1/18

(52) U.S. Cl. 514/1; 435/32

(57) ABSTRACT

The present invention relates to methods of inhibiting the proliferation of a microbial population of a surface lesion of a human or animal, wherein the lesion is contacted with an antimicrobial composition comprising a pharmaceutically acceptable antibiotic, a pharmaceutically acceptable chelating agent and a pharmaceutically acceptable pH buffering agent. Another aspect of the present invention is a medical dressing for delivering an antimicrobial composition to a site of a skin injury or lesion of a mammal, comprising a support and an antimicrobial composition, wherein the antimicrobial composition is at least one antimicrobial agent and at least one chelating agent. The present invention also relates to methods for delivering an antibiotic to an injury to the skin of a human or animal and to kits for the preparation and use of an antimicrobial composition to inhibit a microbial infection of a skin injury or lesion of a human or animal.

The present invention claims the benefit of the provisional U.S. application Serial No. 60/234,375 filed Sep. 20, 2000, which is incorporated herein by reference.

### FIELD OF THE INVENTION

The present invention relates generally to pharmaceutical compositions, medical devices and methods for the administration of the pharmaceutical compositions to inhibit microbial infections of surface wounds or skin lesions of a human or animal. More specifically, the present invention relates to medical dressings that may be applied to burns, ulcers or other lesions of the skin infected by microorganisms. The present invention further relates to washes to inhibit a microbial infection or colonization of the mouth and the skin surfaces, including those of the feet.

### BACKGROUND

The outermost layers of the skin form a physical barrier that protects a human or animal from microbial invasion and the establishment of opportunistic infections. Injuries to the skin may be mechanical, such as an abrasion or cut, a burn from thermal, radiant or chemical exposure or a necrotic lesion of the surface tissue. A break in the cornified layer or epidermis allows microorganisms to penetrate deep into the lower tissues and establish an infection that can spread throughout the body. A burn injury or other skin surface lesion exposes tissue on which a microorganism can thrive. The extent of the infection will depend on the severity or depth of the burn and the surface area affected.

Burns are classified according to the degree of tissue damage. First degree burns are superficial and while the cornified layer may be breached, underlying tissue is not harmed. Second degree burns extend into the epidermal layer and trigger liquid loss with the possibility of shock. Some inhibition of the immune system may also occur. Third degree burns are considerably more severe and extend to the dermis or the underlying tissues. Charring of tissue is evident and there is severe disruption of the immune system.

The combination of a breach in the skin and a compromised immune system means that burn patients are highly susceptible to opportunistic infection of their wounds. Of the nearly two million burn victims per year in the United States, only about seventy thousand are serious enough to require hospitalization. However, as many as ten thousand of these patients will die, usually from a nosocomial infection. Many burn survivors will suffer permanent disfiguring, not just because of scar tissue formation, but also from the tissue damage that accompanies microbial infections. Greater treatment needs and prolongated care of burns patients in the hospital means that infections represent a significant financial drain. Long-term recuperative costs can also be significant.

Ulcers are exposed surface lesions of the skin or a mucoid layer such as the lining of the mouth, where inflamed and necrotic tissue sloughs off. The exposed surfaces of ulcers are highly susceptible to opportunistic microbial invasion. The primary infected site is localized and best treated by a topical application of an antimicrobial agent, perhaps augmented with systemic antibiotic administration. Although less dramatic in terms of morbidity and mortality than burns, infections of surface ulcers and other lesions of the skin and the mouth are nevertheless serious. The injured area is not usually as great as for a burn, but infected ulcers are discomforting to the patient or disfiguring, and even life-threatening if leading to a systemic infection.

Surface infections other than those resulting from physical injury or necrotic lesions of the skin or a mucosal surface are also known. Gingivitis, an infection of the gum of the mouth, dental caries, biofilm coverings on surfaces of teeth, and infections of the outer surface of the eye, for example, represent opportunistic microbial invasions requiring treatment with antimicrobial agents. Biofilms represent a particularly intransigent problem in that bacteria situated therein produce polysaccharide and mucoid coatings resistant to penetration by antibiotics.

Treatment of an infection of a burn or ulcer begins with mechanical cleaning of the wound, usually followed by covering with a bandage, gauze or similar dressing and disinfectant or antibiotic. Preferably the dressing will allow air circulation to prevent the establishment of an anaerobic bacterium and gangrene. Increasingly, however, preventive or active treatments of microbial, particularly nosocomial, infections of skin surface lesions encounter resistance of the infecting microorganism to most commonly available antibiotics and some disinfectants.

Microorganisms associated with nosocomial infections are normal flora in the human intestinal tract. Nosocomial infections, common in compromised patients including burn patients, have increased markedly in recent years. Bacteriemia in patients is especially due to Staphylococcus. aureus, Pseudomonas aeruginosa, Escherichia coli, or Klebsiella sp. and are particularly severe and difficult to treat when caused by methicillin-resistant Staph. aureus, vancomycin-resistant Enterococcus sp., or Ps. aeruginosa. These organisms have been isolated from hospitals, chronic care facilities, and nursing homes. From 1991-1995, the number of bacteremias increased twofold as evidenced by the increased isolation of Enterococcal strains. An increase in incidents of antibiotic resistant strains of Ps. aeruginosa, Enterobacter sp., Klebsiella sp., and Enterococcus sp., has been reported. In particular, Staphylococcus has shown an alarming increase in resistance to oxacillin, and Enterococcus resistance to vancomycin. Bacteria are also becoming resistant to many chemical disinfectants, including quarternary ammonium ions due to the acquisition or activation of genes that encode for efflux pumps. The Arc MDR efflux pump, for example, protects bacteria from a wide spectrum of antibiotics, disinfectants, and organic solvents (Alekshun & Levy).

Gram-negative bacteria cell surfaces are damaged by the exposure to the chelating agent ethylenediaminetetraacetic acid (EDTA) (Roberts et al.). Tris buffer enhances this effect (Goldschmidt & Wyss). Gram-negative bacteria exposed to EDTA-Tris have increased permeability to extracellular solutes and leakage of intracellular solutes, periplasmic enzymes, cell membrane-associated proteins, lipopolysaccharide, protein, and phospholipids (Bayer & Leive; Leive, L.). They are also more sensitive to lysozyme, bactericides and antibiotics (Brown & Richards; Monkhouse & Graves; Russel, A. D.; and Wooley & Jones). The EDTA is a strong chelating agent that removes divalent cations from the bacterial cell wall (Leive et al.), altering the integrity and permeability of the outer membrane. Solutions of EDTA have also been shown to dissolve the biofilm or capsule produced by Ps. aeruginosa (Kreig et al.).

Combinations of EDTA-Tris solutions and selected antimicrobial agents have synergistic antimicrobial effects against Ps. aeruginosa, E. coli and Proteus vulgaris (Farca et al.; Gerberick & Castric; Sparks et al.; Wooley & Jones; Wooley et al. (1983a); Wooley et al. (1984); and Wooley et al. (1983b, c)). But, although combinations of EDTA-Tris with lysozymes or antibiotics are effective in the treatment of coliform infections of the urinary bladder in human patients (Goldschmidt et al.), bacterial rhinitis in dogs (Wooley et al. (1979)), induced Pseudomonas cystitis (Wooley et al. (1974)), otitis externa (Blue et al.; Sparks et al.), and multiple fistulas (Ashworth et al; Bjorling et al.) in dogs, and metritis in the mare (Youngquist, R. S.), all of these treatments typically involve lavage treatment of infections.

Infections of burns, ulcers and other lesions, however, that have penetrated through the skin's cornified layer cannot be washed with lavages without a risk of promoting or spreading the infection, or leaving the wound open for further opportunistic infections. The volumes of lavages that would be used for seriously burned patients would also require large amounts of antibiotics, possibly encouraging antibiotic resistance in populations of microorganisms. Furthermore, seriously burnt patients may not tolerate extensive washing and manipulation of open and tender wound surfaces.

A method of administering an antimicrobial agent to a burn by incorporating the agent into a polymer that degrades and slowly releases the agent to the infected area is disclosed in Schmitt (U.S. Pat. No. 4,122,158). This method, however, does not specifically address the need to enhance the effectiveness of an antibiotic to overcome antibiotic resistance. Yamasaki et al. in U.S. Pat. No. 5,098,417 disclose cellulosic wound dressings with antibacterial and antifungal agents ionically bound thereon. The ionic bonds break when in contact with aqueous bodily exudates to release the biologically active compounds. The wound dressings of Yamazaki et al., however, also do not address the need to enhance antibiotic activity to combat antibiotic resistant strains of bacteria. Raad et al. (U.S. Pat. No. 5,688,516) discloses coating the exterior surface of a catheter device, inserted internally into a patient, with an antibiotic and a chelating agent to prevent infections that may be introduced into the patient by the catheter. There is still a need, however, not addressed in the prior art, for an enhanced antimicrobial composition effective in treating or preventing infections of burns and other skin or surface lesions of humans and animals. Safe and effective means of topically administering such compositions to a skin lesion, such as a burn, that will provide sustainable release of the antimicrobial agent to the wound are also required. The composition should preferably also have enhanced activity against drug resistant strains of infectious microbes.

### SUMMARY OF THE INVENTION

Briefly described, the present invention provides methods and devices for inhibiting an infection of a surface lesion of a human or animal, comprising and contacting a skin injury or surface lesion of a human or animal with an antimicrobial composition comprising a pharmaceutically acceptable antibiotic, a pharmaceutically acceptable chelating agent, and a pharmaceutically acceptable pH buffering agent.

One aspect of the present invention is a medical dressing for delivering an antimicrobial composition to a site of a skin injury or lesion of a human or animal. The dressing comprises a support and an antimicrobial composition, wherein the antimicrobial composition comprises at least one pharmaceutically acceptable antibiotic and at least one pharmaceutically acceptable chelating agent. In one embodiment of the present invention, the therapeutic composition also includes a pH buffering agent that will increase the effective activity of the antibiotic to a microbial infection.

In another embodiment of the present invention, the composition further includes vitamin E that promotes tissue repair and reduces the pain associated with extensive burn injuries.

Another aspect of the present invention relates to methods for delivering an antibiotic to an injury to the skin of a human or animal, and comprises the steps of identifying a site of microbial infection on the surface of a human or an animal, providing a medical dressing for delivering an antimicrobial composition to the site of infection, wherein the medical dressing comprises a support and an antimicrobial composition, the antimicrobial composition comprising at least one pharmaceutically acceptable antibiotic having antimicrobial activity, and at least one pharmaceutically acceptable chelating agent, and a pharmaceutically acceptable pH buffering agent, and applying the medical dressing to the site of infection.

Yet other aspects of the present invention relate to kits for the preparation and use of an antimicrobial composition to inhibit an microbial infection of a skin injury or lesion of a human or animal. Another aspect of the present invention is a kit for the preparation of a medical dressing for the delivery of an antimicrobial composition to a site of infection, and comprises a medical dressing comprising a support, a first vessel containing at least one pharmaceutically acceptable chelating agent, at least one pharmaceutically acceptable antibiotic, at least one pharmaceutically acceptable pH buffering agent and packaging material, with the packaging material including instructions directing the use of the kit for preparing and applying the medical dressing to a human or animal.

The present invention addresses the need for a medical dressing that can be applied to a wound to the skin, especially a burn injury, or a lesion such as an ulcer, and which will deliver a therapeutic amount of an antimicrobial composition that will be effective against infectious microorganisms. The present invention further addresses the need to deliver an effective amount of an antimicrobial composition that will overcome antibiotic resistance of many infectious organisms.

The present invention addresses these needs by providing medical dressings impregnated or having on a surface thereof, a composition comprising at least one antibiotic, a pharmaceutically acceptable pH buffering agent, and at least one chelating agent that increases the sensitivity of a microorganism to the antibiotic, thereby increasing the efficacy of the antibiotic. The present invention further includes a method of applying the medical dressing so as to inhibit the development of a microbial infection. The present invention also addresses the need for a simple method of applying the medical dressing to a wound by supplying a kit containing the dressing and the composition that may be applied to the dressing before it is placed over a skin wound. The dressings and compositions of the present invention are also suitable for application to an injury to the mucosal surface of the mouth, or a tooth to treat or prevent a microbial infection thereof.

Additional objects, features, and advantages of the invention will become more apparent upon review of the detailed description set forth below when taken in conjunction with the accompanying drawing figures, which are briefly described as follows.

### BRIEF DESCRIPTION OF THE FIGURES

FIG. 1. IsoboloGram, illustrating the combined effect of EDTA and neomycin (in 50 mM Tris) on Staphylococcus aureus.

FIG. 2. IsoboloGram, illustrating the combined effect of EDTA and neomycin (in 50 mM Tris) on Pseudomonas aeruginosa.

FIG. 3. IsoboloGram, illustrating the combined effect of EDTA and neomycin (in 50 mM Tris) on Enterococcus faecalis.

FIG. 4. Growth of Staphylococcus aureus on Mueller Hinton agar when treated alone or with combinations of EDTA, water, and neomycin.

FIG. 5. Growth of Pseudomonas aeruginosa on Mueller Hinton agar when treated alone or with combinations of EDTA, water, and neomycin.

FIG. 6. Growth of Enterococcus faecalis on M Enterococcus agar when treated alone or with combinations of EDTA, water, and neomycin.

### DETAILED DESCRIPTION OF THE INVENTION

A full and enabling disclosure of the present invention, including the best mode known to the inventor of carrying out the invention is set forth more particularly in the remainder of the specification, including reference to the Examples. This description is made for the purpose of illustrating the general principles of the invention and should not be taken in the limiting sense.

The present invention addresses the need to deliver an effective dose of an antimicrobial composition of enhanced activity to a skin injury, such as a burn, a skin lesion or a lesion of the oral mucosa. The present invention provides medical dressings that are impregnated, or have on a surface thereof, a therapeutic antimicrobial composition comprising at least one antimicrobial agent a chelating agent, and a pharmaceutically acceptable pH buffering agent. Optionally, the antimicrobial composition may further comprise vitamin E that promotes tissue repair, thereby reducing the likelihood of further opportunistic infection and improved wound healing. The medical dressings of the present invention are useful for application to a burn or other traumatic injury to the skin of a human or animal, or to a skin lesion such as an ulcer or abrasion and which is or may become infected. The antimicrobial agent has increased antimicrobial activity because of the chelating agent and maintenance of the treated area at a pH suitable for sustained antibiotic activity. The antimicrobial agent can therefore be used in effective doses that are less than would be required for the same level of antimicrobial activity in the absence of the chelator. The compositions of the present invention are, therefore, useful in counteracting or preventing an infection or will be more effective against infections caused by drug-resistant strains of microbes.

The present invention further contemplates the use of a composition comprising an antimicrobial agent and a chelating agent in inhibiting a microbial infection or colonization of the oral mucosa. In one embodiment of the present invention the composition is in the form of a mouthwash or mouth rinse that may be contacted with the interior or exterior surfaces of a mouth or buccal cavity. In another embodiment of the present invention, the therapeutic antimicrobial composition impregnates or is on the surface of a medical dressing, including but not limited to, a gauze, a cloth, an absorbent or non-absorbent material that maybe placed onto a site of infection of the mouth. In another embodiment, the therapeutic antimicrobial composition may be mixed with, or otherwise combined with a malleable material such as, but not limited to, a plastic, a resin or any other biologically acceptable material that can be inserted into the mouth for contacting an infected area within.

Definitions

The term “colonization” as used herein refers to the process of a group of bacteria living together. It is further understood that “colonization” may or may not result in a pathological infection.

The term “microbial infection” as used herein refers to any pathological presence of at least one bacterial species on or in an injury or lesion to the skin of a human or animal. It is further understood that a “microbial infection” may include any systemic infection that is amenable to inhibition by application of the antimicrobial compositions of the present invention.

The term “medical dressing” as used herein refers to any covering, protective or supportive, for diseased or injured parts of the skin, or internal organs of a human or animal. The dressing can be, but is not limited to, an absorbent dressing such as a gauze, a sterilized gauze or absorbent cotton, an antiseptic dressing permeated with an antiseptic solution to delay or prevent the onset of an infection, a dry dressing comprising a dry gauze, dry absorbent cotton or any other dry material that may be sterilized by any means known to one of ordinary skill in the art and which does not render the dressing unacceptable for placing over an open wound. The medical dressing as understood by the present invention may also comprise a non-adherent dressing that will not adhere to an infected wound or injury, a protective dressing intended to prevent further injury or infection to the infected part of the body, and a wet dressing wherein the dressing is wetted before application to the infected site. The term “medical dressing” may further include an oil-based support such as vitamin E in which an antimicrobial composition according to the present invention is dissolved. The oil-base such as, for example, vitamin E can form a barrier to further microbial infection and will leach an antimicrobial composition into the damaged tissue.

The term “burn” as used herein refers to tissue injury of the skin caused by thermal, chemical, or radiation exposure or abrasive friction. A burn may be a “first-degree burn” wherein there is superficial damage to the outer cornified layer, a “second-degree burn” wherein the damage extends down into the epidermal layer of cells but is not of sufficient extent that regeneration of the skin is prevented, or a “third-degree burn” wherein the injury extends below the dermis to the underlying tissue and wherein repair of the skin is not possible without grafting. The term “burn” as used herein also refers to any injury to the skin caused by an acid, an alkali, a brush or an abrasion, chemicals, electricity, explosive flash, hot liquids such as, but not only, boiling water, radiant energy such as heat, nuclear radiation or X-rays, or conductive thermal energy transfer due to direct contact with a hot surface or material. The term “burn” as used herein further refers to scalds due to exposure of the skin to hot liquids or gases that result in damage to the skin and underlying tissues.

The terms “lesion” and “surface lesion” as used herein refer to a circumscribed area of pathologically altered tissue, an injury or wound, or a single patch of a skin disease. The term “lesion” as used herein refers to primary lesions which are the immediate result of the pathological condition and may include, but are not limited to, cuts, abrasions, vesicles, blebs, bullae chancres, pustules, tubercles or any other such condition of the skin or a surface of the mouth, nose, anus or any other orifice of the body of a human or animal, or secondary lesions that later develop from a primary lesion and includes, but is not limited to, fissures and ulcers.

The term “ulcer” as used herein refers to an open sore or lesion of the skin or a mucous membrane that involves the sloughing off of inflamed and necrotized tissue and includes, but is not limited to, callous ulcers, chronic leg ulcers, decubitus, denture ulcers of the oral mucosa, traumatic ulcers of the mouth, infections stomatitis of the mouth and any type of secondary lesion that is a breach of the cornified and the epidermal layer of the skin.

The terms “antimicrobial composition”, “antimicrobial agent” and “therapeutic antimicrobial composition” as used herein refer to the compounds and combinations thereof that may be administered to an animal or human and which inhibit the proliferation of a microbial infection.

The term “inhibiting the proliferation of a microbial population” as used herein refers to the bacteristatic or bacteriocidal activity of an antimicrobial composition. A bacteristatic antimicrobial composition will inhibit the multiplication of a bacterial population. A bacteriocidal antimicrobial composition will contact and kill the target microbial population.

The terms “mouthwash” and “mouth rinse” as used herein refer to a medicated solution used to treat or prevent diseases of the mouth or buccal cavity wherein the diseases compromise an infection by a microbial agent.

The term “impregnated” as used herein refers to wetting, soaking or saturating an absorbent material or medical dressing with a liquid. The absorbent material may be, but is not necessarily, dried before application of the dressing to a wound or lesion of the skin or other infected site.

The term “chelating agent” as used herein refers to any organic or inorganic compound that will bind to a metal ion having a valence greater than one, and includes, but is not limited to, organic chelating agents such as ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), and triethylenetetramine hexaacetic acid (TTG), deferoxamine, Dimercaprol, edetate calcium disodium, zinc citrate, penicilamine succimer and Editronate or any other pharmaceutically acceptable chelating agent, salt or combination thereof, known to one of ordinary skill in the art, and which will chelate divalent metal ions such as, but not only Ca²⁺, Mg²⁺, Mn²⁺, Fe²⁺, and Zn²⁺.

The term “antibiotic” as used herein refers to any pharmaceutically acceptable compound known to one of ordinary skill in the art that will inhibit the growth of, or kill, bacteria. The term “antibiotic” includes, but is not limited to, β-lactams penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocins, sulfonamides and trimethoprim, rifampicins, metronidazoles, quinolones, novobiocins, polymixins and Gramicidins and the like and any salts or variants thereof. It also understood that it is within the scope of the present invention that the tetracyclines include, but are not limited to, immunocycline, chlortetracycline, oxytetracycline, demeclocycline, methacycline, doxycycline and minocycline and the like. It is also further understood that it is within the scope of the present invention that aminoglycoside antibiotics include, but are not limited to, gentamicin, amikacin and neomycin and the like.

The term “pH buffering agent” as used herein refers to any pharmaceutically acceptable organic or inorganic compound or combination of compounds that will maintain the pH of an antibiotic-containing solution within 0.5 pH units of a selected pH value. A “pH buffering agent” may be selected from, but is not limited to, Tris (hydroxymethyl) aminomethane (tromethaprim; TRIZMA base), or salts thereof, phosphates or any other buffering agent such as, for example, phosphate-buffered saline that is biologically acceptable.

The term “carrier” as used herein refers to any pharmaceutically acceptable solvent of antibiotics, chelating agents and pH buffering agents that will allow the antimicrobial composition of the present invention to be administered directly to a burn or other skin lesion or to the oral mucosa. The carrier will also allow the composition of the present invention to be applied to a medical dressing for application to the infected region of the skin or mouth of a human or animal. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal. An example of a gel that may be used in compositions of the present invention is KY™ gel (sodium carboxymethylcellulose 7H 4F (Hercules Inc., Wilmington, Del.)). An example of an oil-based carrier is vitamin E.

Microbial species that may cause infections inhibited by the methods of the present invention include bacterial species that may cause infections of a burn or lesion of the skin or oral mucosal lesion of a human or animal include, but are not limited to, Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic E. coli, Enterotoxin-producing E. coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P vulgaris, Pseudomonas aeruginosa, Rhodococcus equi, Salmonella enteridis, S. typhimurium, S. typhosa, Shigella sonnei, S dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The term “biologically active” as used herein refers to the ability of an antibiotic to inhibit the proliferation of a microorganism. The action of the antibiotic can be either bacteriostatic wherein the antibiotic arrests the proliferation of, but does not necessarily kill, the microorganism or the activity of the antibiotic can be bacteriocidal and kill the organism or a combination of activities. Techniques to identify the infecting microorganism and to determine the concentration of the antibiotic that will inhibit or kill fifty percent (MIC₅₀) of the organisms will be considered well known to one of ordinary skill in the art and will not require undue experimentation. The techniques to determine the antibiotic sensitivity of a bacterial isolate, and the methods of determining the synergistic effect of adding a chelating agent to a solution of an antibiotic are described in Manual of Methods for General Microbiology, Eds: Gerhardt et al., American Society of Microbiology, 1981, and incorporated herein in its entirety by reference.

The term “pharmaceutically acceptable” as used herein refers to a compound or combination of compounds that will not damage the physiology of the recipient human or animal to the extent that the viability of the recipient is compromised. Preferably, the administered compound or combination of compounds will elicit, at most, a temporary detrimental effect on the health of the recipient human or animal.

The infection that may be treated by the methods, medical dressings and compositions of the present invention, may be any opportunistic infection by a bacterium, or a multiple infection of more than one species of bacteria, and wherein the proliferation of which can be inhibited by the application of the compositions and medical dressings of the present invention.

The methods and dressings of the present invention may also include tissue growth promoting factors and antioxidants such as vitamin E that are especially useful for treating nosocomial infections of burn patients, and for promoting the healing of the injuries so treated. Tissue growth and repair is promoted, thereby reducing final tissue damage and increasing the acceptance of graft tissue at the injured site, as shown in Example 6, below.

One embodiment of the present invention, therefore, provides a novel method of inhibiting a surface lesion infection of a human or animal, comprising the steps of providing a human or animal having a skin or surface injury or lesion, wherein the injury or lesion can be infected by at least one microorganism, and contacting the skin injury or skin surface lesion with an antimicrobial composition. The antimicrobial composition of the present invention will comprise a pharmaceutically acceptable antibiotic, a pharmaceutically acceptable chelating agent, a pharmaceutically acceptable pH buffering agent and a pharmaceutically acceptable carrier, and which will inhibit the proliferation of a microbe infecting a burn or other lesion of the skin or the oral mucosa of a human or animal.

The therapeutic antimicrobial composition of the present invention may further comprise vitamin E that is useful in reducing the production of toxic compounds at the injury site and for promoting tissue repair.

It is within the scope of the present invention that the antimicrobial composition may be used as a mouth wash or rinse to inhibit the proliferation of infections of lesions of the oral surfaces including the oral mucosa, the teeth, tongue or other surfaces of the oral cavity. The inclusion of a pharmaceutically acceptable chelating agent is useful in attacking biofilms on, for example, teeth that would otherwise prevent access of an antimicrobial agent to the underlying bacteria. It is further contemplated that the antimicrobial composition of the present invention may be used as a bath for the total or partial immersion of a human or animal for the treatment of skin infections or lesions of the skin such as for inhibiting an infection of a foot, or hand, of a human or animal.

Before application of the composition of the present invention, it is anticipated that the identity and the resistance of the infecting microorganism to the antibiotic may be determined by routine tests well known to one of ordinary skill in the art, including determining the MIC and FIC of antibiotics in the absence and/or presence of a chelating agent, and the amount of the antimicrobial composition may be adjusted accordingly so as to inhibit growth of the microorganism. The concentrations and amounts of the antimicrobial agent and chelating agent may be adjusted to levels that are physiologically accepted by the exposed tissue of the injury or lesion and effective against the microbial infection of the skin injury or skin lesion.

In one embodiment of the present invention, the concentration of the antibiotic is in the range of about 0.04 mg/ml to about 25.0 mg/ml and the concentration of the chelating agent in the carrier is in the range of about 0.1 mM to about 100.0 mM.

The present invention further provides a medical dressing comprising a support and a pharmaceutically acceptable antimicrobial composition thereon. The support may be any material that is biologically acceptable and suitable for placing over a wound such as a burn, or a surface lesion of the skin or the oral mucosa or teeth of the mouth. In exemplary embodiments of the present invention, the support may be a woven or non-woven fabric of synthetic or non-synthetic fibers, or any combination thereof. The present invention further contemplates using a support comprising a polymer foam, a natural or man-made sponge, a gel or a membrane that may absorb or have disposed thereon, a therapeutic composition. A gel suitable for use as a support for the antimicrobial composition of the present invention is KY™ (sodium carboxymethylcellulose 7H 4F (Hercules, Inc., Wilmington, Del.)). When vitamin E is included in the antimicrobial composition of the present invention, the oily vitamin E may be used as a solvent of an antimicrobial agent, chelating agent and optionally a pH buffering agent, and may be used to cover a skin injury or lesion.

The present invention contemplates still further that the support may be a film, a natural or synthetic polymer, or a rigid or malleable material that is known to one of ordinary skill in the art as being acceptable for insertion in the mouth of a human or animal, and which will place an antimicrobial composition according to the present invention in contact with a tooth or a lesion of the oral mucosa. In one such embodiment of the medical dressing of the present invention, the support is a gauze. The gauze may be absorbent and can be wetted with an antimicrobial composition of the present invention before applying the gauze to an infected wound or other site.

The present invention also contemplates that the gauze may be impregnated with an antimicrobial composition and then dried. This allows the impregnated dressing to be stored for later use, or to avoid excessively dampening an injured area. In yet another embodiment of the present invention, an antimicrobial composition is absorbed on the surface of the support material of the medical dressing. The composition may be applied to the surface by wetting the surface with a solution of the antimicrobial composition and drying the support to deposit the composition thereon. A concentration of the antimicrobial composition that is effective against the proliferation of a microorganism may be attained when the dressing is wetted by fluids from the patient's injury.

One embodiment of the present invention, therefore, is a gauze impregnated with a pharmaceutically acceptable antimicrobial composition that comprises at least one antimicrobial agent, a chelating agent and a pH buffering agent. Impregnating the absorbent material and placing of the dressing onto a burn or lesion of the skin will allow the antimicrobial agent to be progressively administered to the site of injury. While not wishing to be bound by any one theory, the chelating agent is believed to remove divalent cations from the cell wall of a microbe such as a bacterium, as well as create pores in the bacterial wall. The presence of the chelating agent with the antibiotic increases the effective antimicrobial activity of a particular antibiotic dose.

The selected antibiotic can be effective against opportunistic pathogens infecting a burn, a skin lesion or the oral mucosa and the like. While it is not the intention of the present invention to limit the antimicrobial agent, in various embodiments of the present invention the antimicrobial agent is an antibiotic selected from a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin or a Gramicidin.

In one embodiment, the antibiotic is a penicillin, an aminoglycosides; a vancomycin, a chloramphenicol, an erythromycin, a tetracycline, gentamicin, nalidixic acids, or a streptomycin. In another embodiment the antibiotic is tetracycline. In another embodiment of the present invention, the antibiotic is neomycin. In another embodiment of the present invention, the antibiotic is amikacin. In yet another embodiment, the antibiotic is gentamicin.

The pharmaceutically acceptable chelating agent of the antimicrobial composition applied to the medical dressing of the present invention may be selected from ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate.

In one embodiment of the present invention, the pharmaceutically acceptable chelating agent is selected from ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), and triethylenetetramine hexaacetic acid (TTG).

In other embodiments, the pharmaceutically acceptable chelating agent may be ethylenediamenetetracetic acid (EDTA) or triethylene tetramine dihydrochloride (TRIEN).

The antimicrobial compositions of the present invention may further comprise a pharmaceutically acceptable pH buffering agent that preferably will maintain the pH of the antimicrobial composition, when delivered to the skin injury or skin lesion, to between about pH 7.0 and about pH 9.0. In one embodiment of the present invention, the pH of the antimicrobial composition in solution is about 8.0.

The antimicrobial composition of the present invention may also include vitamin E that will promote tissue growth and repair and reduces the pain experienced at the site of the skin injury. By promoting tissue repair, not only is discomfort to the patient reduced, but there may be less scar tissue formation and hence less permanent disfiguring of the patient. Furthermore, faster healing of a skin injury or lesion promoted by vitamin E is useful in reducing the likelihood of a nosocomial infection and the problems associated therewith.

Another aspect of the present invention is kits that comprise the antimicrobial compositions, or the components to prepare the antimicrobial compositions, and packaging that includes instructions on how to prepare and use the compositions to inhibit the proliferation of a microbial population of a skin injury such as a burn, or of such as a burn, or of a lesion of the skin or the oral mucosa and promote healing thereof. In one embodiment of the present invention, therefore, a kit for preparing the antimicrobial composition of the present invention for inhibiting the proliferation of a microbial infection comprises at least one vessel containing at least one pharmaceutically acceptable chelating agent, at least one pharmaceutically acceptable buffering agent suitable for maintaining the pH of the site of the infection so as to increase the antimicrobial activity of the antimicrobial composition, at least one pharmaceutically acceptable antimicrobial agent and packaging material. The packaging material comprises instructions directing the use of the kit for preparing the antimicrobial composition of the present invention and delivering the composition to a skin injury or skin surface lesion, or to the mouth of a human or animal to inhibit the proliferation of a microbial infection therein. The kit may further comprise a vessel containing vitamin E, and instructions for optionally adding vitamin E to the antimicrobial composition.

Another embodiment of the present invention is a kit for the preparation and use of a medical dressing for the delivery of an antimicrobial composition according to the present invention to a site of a skin injury of a human or animal, comprising a medical dressing, at least one vessel containing at least one pharmaceutically acceptable chelating agent, at least one pharmaceutically acceptable antimicrobial agent, at least one pharmaceutically acceptable buffering agent, and optionally a vessel containing vitamin E and packaging material. The packaging material comprises instructions directing the use of the kit for preparing and applying the medical dressing to a human or animal to inhibit the growth of a microbial infection thereon.

Even though the invention has been described with a certain degree of particularity, it is evident that many alternatives, modifications, and variations will be apparent to those skilled in the art in light of the present disclosure. Accordingly, it is intended that all such alternatives, modifications, and variations that fall within the spirit and the scope of the invention be embraced by the defined claims.

The following examples are presented to describe preferred embodiments and utilities of the present invention, but should not be construed as limiting thereof.

It should be appreciated by those of skill in the art that the techniques disclosed in the following examples represent techniques discovered by the inventors to function well in the practice of the invention, and thus can be considered to constitute preferred modes for its practice. Those of skill in the art, however, in light of the present disclosure, will appreciate that many changes can be made in the specific embodiments disclosed and still obtain like or similar results without departing, again, from the spirit and scope of the present invention.

### EXAMPLE 1

### Determination of Synergistic Actions and Fractional Inhibitory Concentration (FIC) Index

The antibacterial action of combinations of EDTA-Tris and neomycin was measured by a two-dimensional microtiter checkerboard technique described in Gilman et al., The Pharmacological Basis of Therapeutics, eds Goodman and Gilman, 1085-1086 (Macmillan Publishing Co., New York, 1985),. Sabath, L. D, Antimicrob. Agents and Chem. 210-217. (1967) and Sparks et al., Vet. Res. Comm.. 18, 241-249 (1994), incorporated herein by reference in their entireties.

Each well of a round-bottomed 96-well microtiter plate was inoculated with 0.05 ml of 2-fold dilutions of neomycin, and of EDTA in 50 mM Tris. Then 0.05 ml of an 18-hour old culture of a test organism, containing 10⁶ colony-forming units (CFU)/ml, were added to each well. Controls for the culture and media were included in each plate. Plates were covered and incubated at 37° C. for 18-24 hours.

Results were plotted as isobolograms for the determination of antagonistic, neutral or additive, or synergistic effects. To generate isobolograms, FICs of the two test solutions were plotted individually on the x-axis and y-axis to determine the effect of combining the two test solutions on bacterial growth. A line that curves away from the zero point and the coordinates indicates antagonism. A straight line indicates neutral or additive effects. Lines that curves toward the zero point and the coordinates are indicative of synergism if there is at least a 4-fold decrease in the MIC of each compound, when used in combination, as compared with the MIC of each test compound alone as described in Gilman et al., The Pharmacological Basis of Therapeutics, eds Goodman and Gilman, 1085-1086 (Macmillan Publishing Co., New York, 1985),. Sabath, L. D, Antimicrob. Agents and Chem. 210-217. (1967), and incorporated herein by reference in their entireties.

A numerical score or fractional inhibitory concentration (FIC) index was determined. The FIC index is equal to the sum of the values of FIC for the individual drugs:

$$FIC = \frac{MIC o f Drug A with Drug B}{MIC of Drug A} + \frac{MIC of Drug B with Drug A}{MIC of Drug B}$$

An FIC index greater than 1.0 indicates an antagonistic interaction, an FIC index of 1.0 indicates addition, and an FIC index of less than or equal to 0.5 indicates synergism between the two test agents.

### EXAMPLE 2

### Inhibition of the Growth of Microorganisms Infecting Burns

The organisms of this study were isolated from human burn patients. They included strains of methicillin resistant Staphylococcus aureus, and vancomycin resistant strains of Pseudomonas aeruginosa, and Enterococcus faecalis. The bacterial isolates were propagated in or on Brain Heart Infusion broth (BHI), Mueller-Hinton Broth (MHB), blood agar (BA), Mueller-Hinton agar (MHA), enterococcus agar (EA), or 2X nutrient agar (2xNA).

The EDTA-Tris treatment solutions were prepared from a stock solution containing 0.5 mols/l sodium EDTA and 1.0 mols/l Tris-HCl, pH 8.0. The treatment solutions contained 5 mM sodium EDTA and 50 mM Tris-HCl with or without of neomycin sulfate 1 mg/ml.

Antibiotic resistance profiles were determined by the disc diffusion method on MHA (5). Antibiotics tested included ampicillin (AM-10), chloramphenicol (C-30), ciprofloxacin (CIP-5), kanamycin (K-30), gentamicin (GM-10), nalidixic acid (NA-30), neomycin (N-30), streptomycin (S-10), sulfisoxazole (G-25), tetracycline (Te-30), and vancomycin (Va-30).

Minimal Inhibitory Concentrations (MICs) and Minimal Bactericidal Concentrations (MBCs) for EDTA-Tris and neomycin were determined by the broth-dilution microtiter method in MHB or BHI according to the method of Blair et al., Manual of Clinical Microbiology. p.307 (pub: Am. Soc. Microbiol. Williams and Wilkins, Baltimore 1970), incorporated herein by reference in its entirety.

### EXAMPLE 3

### In Vitro Effect of EDTA-Tris and Neomycin on Enterococcus faecalis, Pseudomonas aeruginosa, and Staphylococcus aureus

2xNA plates were swabbed with 200 ml of an overnight culture containing about 10⁷ colony-forming-units of a test organism. The plates were sampled with multipoint contactors as described in Wooley et al., Am. J. Vet. Res. 35, 807-810. (1974), incorporated herein by reference in its entirety. Each multipoint contactor consisted of an array of 27 mm sewing needles mounted to an aluminum plate measuring 1 mm×50 mm×50 mm. The needles were set 3.5 mm apart. The multipoint contactors were sterilized by autoclaving. To collect samples, a multipoint contactor was touched to an overnight bacterial culture grown on 2xNA as described above. Replicate plates were then inoculated by lightly pressing the needles bearing the test bacteria onto either MHA plates, BA plates or EA plates for Ps. aeruginosa, Staph. aureus and Ent. faecalis respectively. The agar plates were incubated at 37° C. and colonies were counted at 24 hours and 48 hours.

Each strain of microorganism was tested on a control agar plate (plate 1), and on plates wherein the inoculated bacteria were covered with a sterile surgical gauze saturated with 7 ml of: 5 mM EDTA-Tris (plate 2); 5 mM EDTA-Tris and 1 mg/ml neomycin (plate 3); 1 mg/ml neomycin (plate 4); sterile water (plate 5). Samples were taken at 0 mins, and at 30 mins, 1 hour, 2 hours, 4 hours, 6 hours, 8 hours, and 24 hours of incubation.

### EXAMPLE 4

### The Antibiotic Resistance Profiles, MIC and MBC Values for Test Strains of Staph. aureus, Ps. aeruginosa, and Ent. faecalis

The antibiotic resistance profiles and MIC values for test strains of Staph. aureus, Ps. aeruginosa, and Ent. faecalis are shown on Table 1.

TABLE 1
Antibiotic resistance profiles of Staphylococcus aureus, Pseudomonas
aeruginosa, and Enterococcus faecalis.

| | Antimicrobial Agents^{A} | | | | | | | | | | |
|---|---|---|---|---|---|---|---|---|---|---|---|
| | Am | C | Cip | Gm | K | NA | N | S | G | Te | Va |
| Staphylococcus aureus | R^{B} | I | R | S | R | R | R | S | S | S | S |
| Pseudomonas aeruginosa | R | R | I | I | R | R | R | R | R | R | R |
| Enterococcus faecalis | S | R | R | R | R | R | R | R | R | R | R |

^{A}Am = ampicillin; C = chioramphenicol; Cip = ciprofloxacin; K = kanamycin; Gm = gentamicin; NA = nalidixic acid; N = neomycin; S = streptomycin; G = sulfisoxazole; Te = tetracycline; Va = vancomycin; ^{B}R = resistant; I = intermediate; S = sensitive.

Fractional inhibitory concentrations (FICs) and isoboloGrams for the EDTA-Tris, neomycin combination to determine a synergistic, additive, or antagonistic reaction, as described in Example 1, were determined for Staph. aureus, Ps. aeruginosa, and Ent. faecalis. MIC and MBC values for concentrations of neomycin, ampicillin, chloramphenicol, amikacin and oxytetracycline and EDTA administered individually, and the FIC values for Staph. aureus, Ps. aeruginosa, and Ent. faecalis are shown in Table 2 (Columns 2 and 3). MIC values for mixtures of the above antibiotics and EDTA in the presence of each other are shown in Table 2 (Columns 4 and 5 respectively).

TABLE 2
Minimal Inhibitory Concentration (MIC) data concerning the amounts (mM)
of EDTA in 50 mM Tris and antibiotics (mg/ml) when reacting alone and in
combination against Pseudomonas aeruginosa, Staphylococcus aureus, and
Enterococcus faecalis.

| | MIC | | | | |
|---|---|---|---|---|---|
| | Individually | | | | |
| | Administered | | Co-administered | | |
| | Neomycin | EDTA | Neomycin + EDTA | | FIC |
| Ps. aeruginosa | 1.0 | 1.25 | 0.063 | 0.156 | 0.19 |
| Staph. aureus | 3.13 | 1.0 | 1.56 | 0.25 | 0.75 |
| Ent. faecalis | 3.13 | 15.63 | 1.17 | 1.88 | 0.5 |
| | Ampicillin | EDTA | Ampicillin + EDTA | | FIC |
| Ps. aeruginosa | 0.49 | 1.25 | 0.123 | 0.156 | 0.38 |
| Staph. aureus | 0.24 | 1.0 | 0.0075 | 0.25 | 0.28 |
| Ent. faecalis | 0.001 | 15.63 | 0.00025 | 7.82 | 0.75 |
| | Chloramphenicol | EDTA | Chloramphenicol + EDTA | | FIC |
| Ps. aeruginosa | 12.5 | 1.25 | 1.56 | 0.313 | 0.37 |
| Staph. aureus | 0.39 | 1.0 | 0.39 | 1.0 | 2.0 |
| Ent. faecalis | 0.4 | 15.63 | 0.2 | 3.9 | 0.75 |
| | Amikacin | EDTA | Amikacin + EDTA | | FIC |
| Ps. aeruginosa | 0.001 | 1.25 | 0.001 | 1.25 | 2.0 |
| Staph. aureus | 0.12 | 1.0 | 0.03 | 0.5 | 0.75 |
| Ent. faecalis | 2.0 | 15.63 | 1.0 | 7.8 | 1.0 |
| | Oxytetracycline | EDTA | Oxytetracycline + EDTA | | FIC |
| Ps. aeruginosa | 0.003 | 1.25 | 0.00075 | 0.313 | 0.5 |
| Staph. aureus | 0.0001 | 1.0 | 0.00005 | 0.5 | 1.0 |
| Ent. faecalis | 0.05 | 15.63 | 0.025 | 3.91 | 0.75 |

*Synergistic reaction (FIC =≦ 0.5)
Additive reaction (FIC => .05 to ≦ 1.0)
Antagonistic reaction (FIC => 1.0)

The MBC values for EDTA and neomycin were decreased by at least 75% for bacterial killing (MBC) in those situations in which synergistic potentation occurred (Ps. aeruginosa and Ent. faecalis) as shown in Table 3. A decrease of about 50% was observed with Staph. aureus.

TABLE 3
Minimal Bactericidal Concentrations (MBC), of Staphylococcus aureus,
Pseudomonas aeruginosa, and Enterococcus faecalis reacted with
EDTA (mM) and neomycin (mg/ml) in 50 mM Tris.

| | | Individually | |
|---|---|---|---|
| Bacterial Species | | Administered | Co-administered |
| Staphylococcus aureus | EDTA | 7.81 | 3.9 |
| | (mM) | | |
| | Neomycin | 3.13 | 1.56 |
| | (mg/ml) | | |
| Pseudomonas aeruginosa | EDTA | 250 | 20.0 |
| | (mM) | | |
| | Neomycin | 5.0 | 0.04 |
| | (mg/ml) | | |
| Enterococcus faecalis | EDTA | 250 | 62.5 |
| | (mM) | | |
| | Neomycin | 25.0 | 6.25 |
| | (mg/ml) | | |

Specifically in the case of Staph. aureus, the MBC values for EDTA and neomycin when combined were decreased by 50% as compared to the bactericidal effect of each when individually administered.

With Ps. aeruginosa, the MBC values for EDTA and neomycin when in combination were decreased 99.2% compared to when EDTA or neomycin were individually administered. In the case of Ent. faecalis, MBC values of EDTA and neomycin were both reduced 75% compared to when EDTA and neomycin were administered individually.

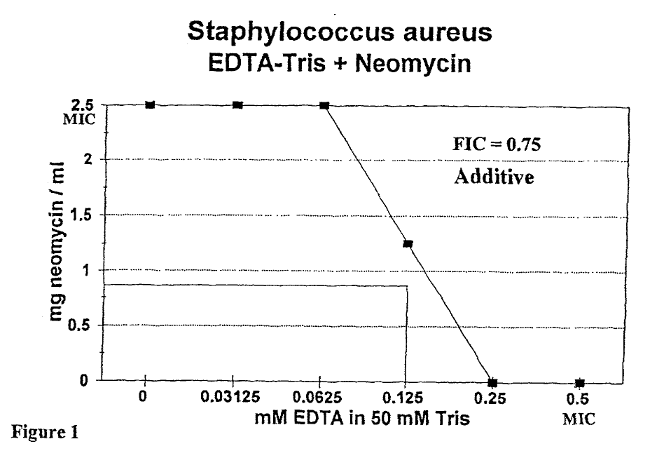

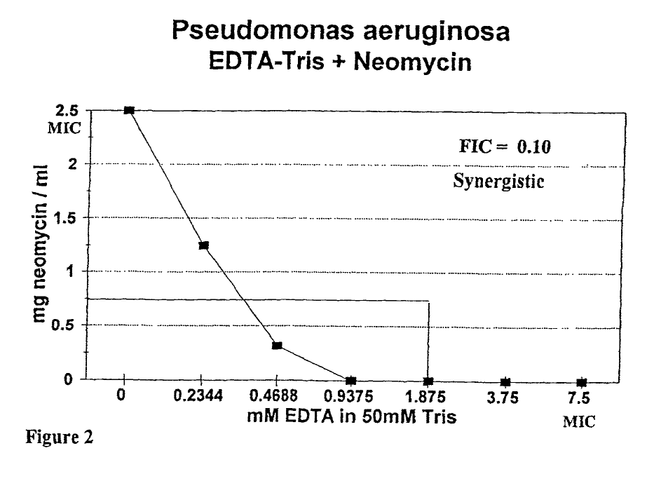

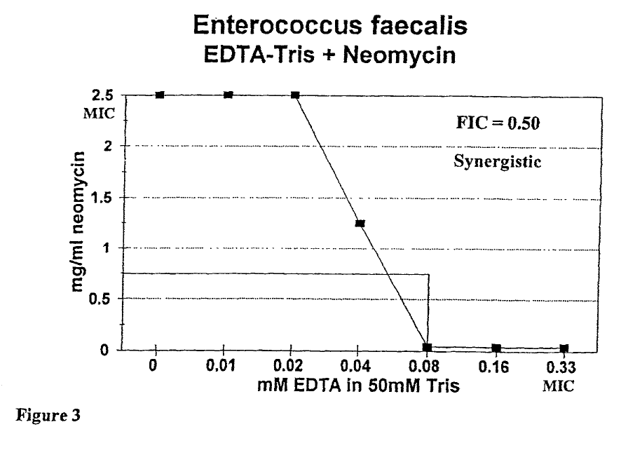

Synergistic effects were observed when various concentrations of EDTA-Tris and neomycin were reacted with Ps. aeruginosa and Ent. faecalis, while an additive effect was observed with Staph. aureus as shown in FIGS. 1-3.

### EXAMPLE 5

### Inhibition of Growth of Ps. aeruginosa and Staph. aureus

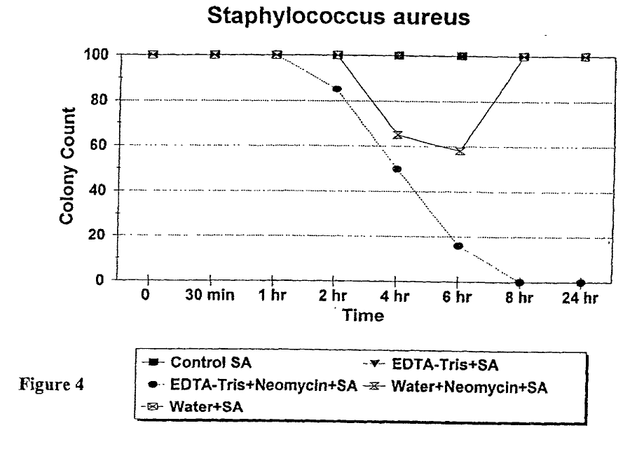

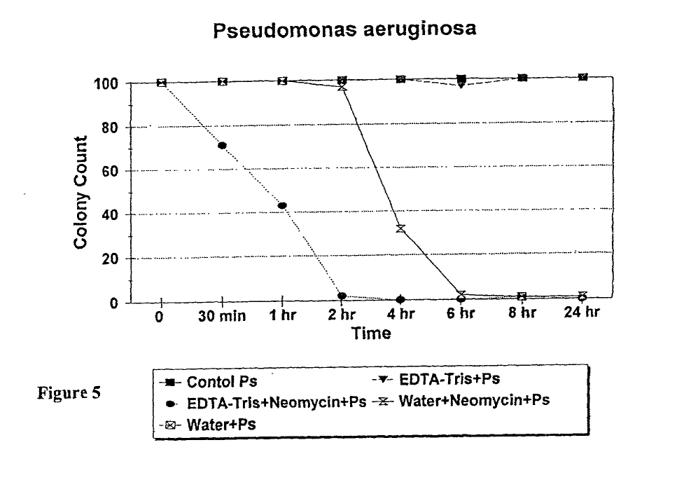

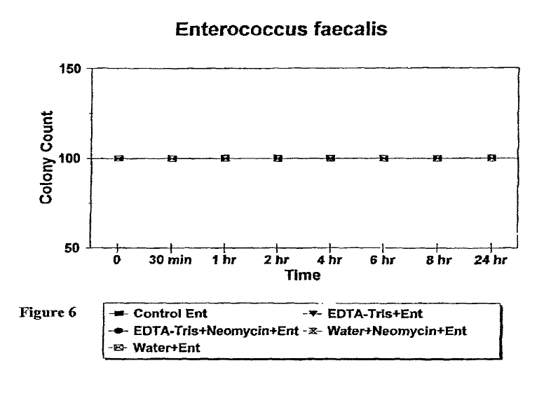

In the in vitro gauze-point-contactor study, the potentation effect was seen for EDTA-Tris-neomycin reactions with Ps. aeruginosa and Staph. aureus. These reactions are illustrated in FIGS. 4 and 5. When the same combinations of EDTA-Tris and neomycin were reacted with Ent. faecalis, no antibacterial activity was noted at these concentrations as shown in FIG. 6.

### EXAMPLE 6

### Treatment of a Skin Burn of a Dog and Antimicrobial Protection of Graft Donor Sites by Vitamin E with EDTA-Tris and Antibiotic

A mixed-breed, 35 pound spayed female canine, 1-2 years old had been doused with gasoline and set on fire and severely burned. The dog was given initial emergency treatment for 5 days. The burned area over 30% of body was cultured for microbial infection and the following bacteria were identified: β-hemolytic Escherichia coli, Klebsiella oxytoca, Proteus sp., and Enterococcus sp. The dog was administered cefazolin systemically and the burned area was cleared of tissue debris and wetted with a solution of EDTA-Tris and neomycin daily. The burn area was free of the four bacteria after 3 days of systemic and topical EDTA-Tris-neomycin therapy. After approximately 10 days, neomycin was replaced with amikacin. The dog received an autologous skin graft approximately three weeks after the burning incident and the donor site was treated with a compound consisting of EDTA-Tris-amikacin and 100 IU of vitamin E. The dog was discharged from veterinarian hospital care two weeks later.

### EXAMPLE 7

### Treatment of Microbially Infected Skin and Oral Lesions

A composition comprising EDTA, Tris and neomycin in KY™ gel carrier was applied to skin ulcers of a turtle, a snake and a frog. Infection was reduced until eliminated, and the treated animals fully healed of their injuries and infections.

A 13 year old domestic short hair cat had developed proliferative gingivitis. The mouth was swabbed with a cotton-tipped swab twice daily for a week with a solution containing 5 mM EDTA, 50 mM Tris, and 2 mg/ml neomycin. After the first week, the mouth and gums were swabbed twice weekly for a further month. Following clearance of the infection from the animal's mouth, there was no recurrence for at least one year. A similar human oral lesion also responded to this treatment. Likewise, mouthwashes also containing EDTA, Tris and neomycin, as above, were used to treat and heal infections stomatitis of the oral cavities of iguanas and snakes.

### EXAMPLE 8

### Vitamin E Does Not Reduce the Antibacterial Efficacy of EDTA-Tris and Antibiotics

The addition of vitamin E to solutions of EDTA-Tris or EDTA-Tris plus antibiotics did not decrease the efficacy of the antibacterial action. The addition of vitamin E to solutions of EDTA-Tris, however, enhanced the antibacterial effect of the solution. Solutions of vitamin E alone decreased the numbers of Ps. aeruginosa significantly, as shown in Table 4.

TABLE 4
Effect of Vitamin E on the Efficacy of EDTA-Tris and Antibiotics when
Tested against Pseudomonas aeruginosa.

| Groups | Log₁₀ Colony-Forming-Units/ml |
|---|---|
| PBS | 8.20 |
| EDTA-Tris | 3.30 |
| EDTA-Tris + Neomycin | No growth |
| EDTA-Tris + Amikacin | No growth |
| Vitamin E | 6.04 |
| Vit E + EDTA-Tris | No growth |
| Vit E + EDTA-Tris + Neomycin | No growth |
| Vit E + EDTA-Tris + Amikacin | No growth |
| Vit E + Neomycin | 4.40 |
| Vit E + Amikacin | No growth |

### EXAMPLE 9

### Stability and Antibacterial Activity of EDTA-Tris-Neomycin Solution (ETN) in KY Gel

Mixtures were prepared, stored at room temperature, and tested monthly against the test organism Pseudomonas aeruginosa. Compositions were prepared with or without KY™ gel. KY™ gel was Sodium Carboxymethylcellulose 7H 4F (Food Grade) (Hercules, Inc., Wilmington, Del.).

Addition of 1% or 2% KY™ gel to the EDTA-Tris-antibiotic compositions did not affect the long term stability and antibacterial activity of the solutions of EDTA-Tris and neomycin, as shown in Table 5.

TABLE 5
Effect of mixing KY ™ gel on EDTA-Tris-neomycin (ETN) storage
stability.
Log₁₀ CFU/ml

| | | | | ETN + 2% KY |
|---|---|---|---|---|
| | PBS | ETN | ETN + 1% KY gel | gel |
| 1 month | 7.60 | NG | NG | NG |
| 2 months | 7.56 | NG | 4.04 | 4.48 |
| 3 months | 6.78 | NG | NG | 2.00 |
| 4 months | 7.73 | NG | NG | NG |
| 5 months | 6.64 | NG | NG | NG |
| 6 months | 6.94 | NG | NG | NG |
| 7 months | 7.40 | NG | NG | NG |
| 8 months | 7.56 | NG | NG | NG |
| 9 months | 8.00 | NG | NG | NG |
| 10 months | 7.85 | NG | NG | NG |

NG = no growth

### REFERENCES

The following references are specifically incorporated herein by reference for the purposes indicated:

Alekshun, M. N. & Levy, S. B. Regulation of Chromosomally mediated Multiple Antibiotic Resistance: The mar Regulon. Antimicrob. Agents & Chemotherapy 41, 2067-2075 (1997).

Ashworth, C. D. & Nelson D. R. Antimicrob. Potentiation of Irrigation Solutions Containing Tris-(hydroxymethyl) aminomethane-EDTA. J. Am. Vet. Med. Assoc. 197, 1513-1514. (1990).

Bayer, M. E. & Leive L. Effect of Ethlyenediamintetraacetate Upon the Surface of Escherichia coli. J. Bacteriol. 130, (1364-1381. 1977).

Bjorling, D. E. &. Wooley R. E. EDTA-Tromethamine Lavage as an Adjunct Treatment for Multiple Fistulas in a Dog. J. Am. Vet. Med. Assoc. 181, 596-597. (1982).

Blue, J. L., Wooley R. E. & Eagon, R. G. Treatment of Experimentally Induced Pseudomonas aeruginosa Otitis Externa in the Dog by Lavage with Ethylenediaminetetracetate-Tris (hydroxymethyl) Aminomethane Lysozyme. Am. J. Vet. Res. 35, 1221-1223. (1974).

Brown, M. R. W. & Richards, M. E. Effect of Ethylenediaminetetraacetate on the resistance of Pseudomonas aeruginosa to antibacterial agents. Nature (London). 207, 1391-1393. (1965).

Farca, A. M., Nebbia, P. & Re, G. Potentiation of the In Vitro Activity of Some Antimicrobial Agents against Selected Gram-Negative Bacteria by EDTA-Tromethamine. Vet. Res. Comm. 17, 77-84. (1993).

Gerberick, G. F. & Castric, P. A. In vitro Susceptibility of Pseudomonas aeruginosa to Carbenicillin, Glycine, and Ethylenediaminetetraacetic Acid Combinations. Antimicrob. Agents & Chemotherapy. 17, 732-735. (1980).

Goldschmidt, M. C., Kuhn, C. R., Perry, K. & Johnson, D. E. EDTA and Lysozyme Lavage in the Treatment of Pseudomonas and Coliform Bladder Infections. J. Urol. 107, 969-972. (1972).

Goldschmidt, M. C. & Wyse, O. The role of Tris in EDTA Toxicity and Lysozyme Lysis. J. Gen. Microbiol. 47, 421-431 (1967).

Kreig, D. P., Bass, .A. & Mattingly, S. J. Phosphorylcholine stimulates Capsule Formation of Phosphate-Limited Mucoid Pseudomonas aeruginosa. Infect. Immun. 56, 864-873 1988).

Leive, L. A Nonspecific Increase in Permeability in Escherichia coli Produced by EDTA. Proc. Nat. Acad. Sci. USA. 53, 745-750 (1968).

Leive, L., Shovlin, V. K. & Mergenhagen, S. E. Physical, Chemical, and Immunological Properties of Lipopolysaccharide Released from Escherichia coli by Ethylenediaminetetraacetate. Biol. Chem. 243, 6384-6391 (1968).

Monkhouse, D. C. & Graves, G. A. The Effect of EDTA on the Resistance of Pseudomonas aeruginosa to Benzalkonium, Chloride. Aust. J. Pharm. 48, 570-575 (1967).

Roberts, N. A., Gray, G. W. & Wilkinson, S. C. The Bactericidal Action of Ethylenediamine-tetra-acetlc Acid on Pseudomonas aeruginosa. Microbios 7-8, 189-208. (1970).

Russel, A. D. Effect of Magnesium Ions & Ethylenediaminetetraacetic acid on the Activity of Vancomycin against Escherichia coli and Staphylococcus aureus. J. Appl. Bacteriol. 30, 395-401 (1967).

Sabath, L. D. Synergy of Antibacterial Substances by Apparently Known Mechanisms. Antimicrob. Agents & Chemotherapy. 210-217 (1967).

Sparks, T. A., Kemp, D. T., Wooley R. E. & Gibbs, P. S. Antimicrobial Effect of Combinations of EDTA-Tris and Amikacin or Neomycin on the Microorganisms Associated with Otitis Externa in Dogs. Vet. Res. Comm. 18, 241-249 (1994).

Wooley, R. E., Berman, A. P. & Shotts Jr, E. B. Antibiotic-Tromethamine-EDTA Lavage for the Treatment of Bacterial Rhinitis in a Dog. J. Am. Vet. Med. Assoc. 75, 817-818 (1979).

Wooley, R. E. & Blue, J. L. In Vitro Effect of EDTA-Tris-Lysozyme on Selected Pathogenic Bacteria. J. Med. Microbiol. 8, 189-194 (1975).

Wooley, R. E., Blue, J. L., Scott, T. A. & Belcher, M K. Attempt to Induce Pseudomonas pyoderma in the Dog. Am. J. Vet. Res. 35, 807-810 (1974).

Wooley, R. E., Dickerson, H. W., Siramens, K. W., Shotts Jr., E. B. & Brown, J. Effect of EDTA-Tris on an Escherichia coli Isolate Containing R Plasmids. Vet. Microbiol. 12, 65-75 (1986).

Wooley, R. E. & Jones, M. S. Action of EDTA-Tris and Antimicrobial Agent Combinations on Selected Pathogenic Bacteria. Vet. Microbiol. 8, 271-280 (1983).

Wooley, R. E., Jones, M. S. & Shotts Jr., E. B. Uptake of Antibiotics in Gram-negative Bacteria Exposed to EDTA-Tris. Vet. Microbiol. 10, 57-70 (1984).

Wooley, R. E., Jones, M. S., Gilbert, J. P. & Shorts Jr., E. B. In Vitro Action of Combinations of Antimicrobial Agents and EDTA-Tromethamine on Escherichia coli. Am. J. Vet. Res. 44, 1154-1158 (1983a).

Wooley, R. E., Jones, M. S., Gilbert, J. P. & Shotts Jr., E. B. In Vitro Action of Combinations of Antimicrobial Agents with EDTA-Tromethamine on Proteus vulgaris of Canine Origin. Am. J. Vet. Res. 45, 1451-1454 (1984).

Wooley, R. E., Jones, M. S., Gilbert J. P., & Shotts Jr., E. B. In Vitro Action of Combinations of Antimicrobial Agents and EDTA-Tromethamine on Pseudomonas aeruginosa. Am. J. Vet. Res. 44, 1521-1524 (1983b).

Wooley, R. E., Jones, M. S., Gilbert J. P., & Shotts Jr., E. B. In Vitro Effect of Combinations of Antimicrobial Agents and EDTA-Tromethamine on certain Gram-positive Bacteria. Am. J. Vet. Res. 44, 2167-2169 (1983c).

Wooley, R. E., Schall, W. D., Eagon, R. G. & Scott, A. A. S. Efficacy of EDTA-Tris-Lysozyme Lavage in the Treatment of Experimentally Induced Pseudomonas aeruginosa Cystitis in the Dog. Am. J. Vet. Res. 35, 27-29 (1974).

Youngquist, R. S. Pseudomonas metritis in a mare. Vet. Med./Small An. Clinician 70, 340-342 (1975).

Schmitt (U.S. Pat. No. 4,122,158)

Yamazaki et al. (U.S. Pat. No. 5,098,417)

Raad et al. (U.S. Pat. No. 5,688,516)

What is claimed is:

1. A method of inhibiting the proliferation of a microbial population or microbial colonization of a human or animal patient, comprising the steps of:
(a) providing the human or animal patient having a skin injury or a surface lesion;
(b) contacting the skin injury or the surface lesion with an antimicrobial composition, wherein the composition comprises a pharmaceutically acceptable antimicrobial agent, a pharmaceutically acceptable chelating agent, a pharmaceutically acceptable pH buffering agent, and a pharmaceutically acceptable carrier; and
(c) inhibiting the proliferation of a microbial population of the skin injury or the surface lesion of the human or animal patient.

2. The method of claim 1, further comprising the steps of:
(a) identifying the microbial population;
(b) identifying an antibiotic capable of inhibiting the proliferation of the microbial population;
(c) determining the MIC and FIC values for the antibiotic and the chelating agent; and
(d) adjusting the concentration of the antibiotic and the chelating agent of the antimicrobial composition to inhibit the proliferation of the microbial population

3. The method of claim 1, wherein the pharmaceutically acceptable chelating agent is selected from the group consisting of ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate.

4. The method of claim 1, wherein the pharmaceutically acceptable chelating agent is further selected from the group consisting of ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), and triethylenetetramine hexaacetic acid (TTG).

5. The method of claim 1, wherein the pharmaceutically acceptable chelating agent is ethylenediamenetetracetic acid (EDTA).

6. The method of claim 1, wherein the pharmaceutically acceptable chelating agent is triethylene tetramine dihydrochloride (TRIEN).

7. The method of claim 1, wherein the pharmaceutically acceptable antimicrobial agent is an antibiotic selected from the group consisting of a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a Gramicidin.

8. The method of claim 7 wherein the pharmaceutically acceptable antimicrobial agent is further selected from the group consisting of a β-lactam, an aminoglycoside, a vancomycin, a chloramphenicol, an erythromycin, a tetracycline, gentamicin, nalidixic acid and a streptomycin.

9. The method of claim 1, wherein the pharmaceutically acceptable antimicrobial agent is oxytetracycline.

10. The method of claim 1, wherein the pharmaceutically acceptable antimicrobial agent is amikacin.

11. The method of claim 1, wherein the pharmaceutically acceptable antimicrobial agent is neomycin.

12. The method of claim 1, wherein the pharmaceutically acceptable antimicrobial agent is biologically active against a Gram-negative bacterial species.

13. The method of claim 1, wherein the pharmaceutically acceptable antimicrobial agent is biologically active against a Gram-positive bacterial species.

14. The method of claim 1, wherein the microbial population is a bacterial infection comprising at least one Gram-negative bacterial genus selected from the group consisting of Aeromonas, Pseudomonas, Escherichia, Enterococcus, Yersinia, Vibrio, Flexibacter, Nocardia, Flavobacterium, Edwardsiella and Cytophagia.

15. The method of claim 1, wherein the microbial population is a bacterial infection comprising at least one Gram-positive bacterial genus selected from the group consisting of Bacillus, Staphylococcus, Streptococcus, and Mycobacterium.

16. The method of claim 1, wherein the pharmaceutically acceptable pH buffering agent comprises Tris (hydroxymethyl) aminomethane (TRIZMA Base).

17. The method of claim 1, wherein the antimicrobial composition further comprises vitamin E.

18. The method of claim 1, wherein the skin injury is a burn.

19. The method of claim 1, wherein the skin injury is an abrasion.

20. The method of claim 1, wherein the skin injury is an ulcer.

21. The method of claim 1, wherein the surface lesion is a lesion of the oral mucosa of a human or animal patient.

22. The method of claim 1, wherein the antimicrobial composition is a mouthwash for inhibiting the proliferation of a microbial population of the oral cavity of a human or animal.

23. A medical dressing for delivering an antimicrobial composition to a skin injury or lesion in a human or animal, comprising:
a support; and
an antimicrobial composition comprising at least one pharmaceutically acceptable antimicrobial agent, at least one pharmaceutically acceptable chelating agent, and at least one pharmaceutically acceptable pH buffering agent.

24. The medical dressing of claim 23, wherein the support is sterile.

25. The medical dressing of claim 23, wherein the support is selected from the group consisting of woven fabrics of naturally occurring fibers, woven fabrics of man-made fibers, non-woven fabrics of naturally occurring fibers, non-woven fabrics of man-made fibers, strands of naturally occurring fibers, strands of man-made fibers, interconnected strands of man-made fibers and interconnected strands of naturally occurring fibers, polymer foams, naturally occurring or synthetic sponges, a biologically acceptable gel, and a membrane.

26. The medical dressing of claim 23, wherein the support is a gauze material.

27. The medical dressing of claim 23, wherein the support is a physiologically acceptable gel.

28. The medical dressing of claim 23, wherein the membrane is a monolayer or a laminate, and wherein the membrane is comprised of a non-synthetic material, a synthetic material, or a combination thereof.

29. The medical dressing of claim 23, wherein the antimicrobial composition impregnates the support.

30. The medical dressing of claim 23, wherein the antimicrobial composition forms at least one layer on at least one surface of the support.

31. The medical dressing of claim 23, wherein the support selected from the group consisting of a film, a moldable plastic or resin, gauze, and fabric, and wherein the support is suitable for contacting a surface lesion in the mouth of a human or animal.

32. The medical dressing of claim 23, wherein the pharmaceutically acceptable chelating agent is selected from the group consisting of ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate.

33. The method of claim 23, wherein the pharmaceutically acceptable chelating agent is further selected from the group consisting of ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), and triethylenetetramine hexaacetic acid (TTG).

34. The medical dressing of claim 23, wherein the pharmaceutically acceptable chelating agent is ethylenediamenetetracetic acid (EDTA).

35. The medical dressing of claim 23, wherein the pharmaceutically acceptable chelating agent is triethylene tetramine dihydrochloride (TRIEN).

36. The medical dressing of claim 23, wherein the pharmaceutically acceptable antimicrobial agent is an antibiotic selected from the group consisting of a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a sreptomycin, a rifampicin, a metronidazole, a quinolone, a novobiocin, a polymixin and a Gramicidin.

37. The medical dressing of claim 23, wherein the pharmaceutically acceptable antimicrobial agent is further selected from the group consisting of a penicillin, an aminoglycoside; a vancomycin, a chloramphenicol, an erythromycin, a tetracycline, gentamicin, nalidixic acid; and a streptomycin.

38. The medical dressing of claim 23, wherein the pharmaceutically acceptable antimicrobial agent is oxytetracycline.

39. The medical dressing of claim 23, wherein the pharmaceutically acceptable antimicrobial agent is selected from neomycin, amikacin and gentamicin.

40. The medical dressing of claim 23, wherein the pharmaceutically acceptable antimicrobial agent is neomycin.

41. The medical dressing of claim 23, wherein the pharmaceutically acceptable antimicrobial agent is amikacin.

42. The medical dressing of claim 23, wherein the pharmaceutically acceptable antimicrobial agent is biologically active against Gram-negative bacterial species.

43. The medical dressing of claim 23, wherein the pharmaceutically acceptable antimicrobial agent is biologically active against a Gram-positive bacterial species.

44. The medical dressing of claim 23, wherein the pharmaceutically acceptable pH buffering agent comprises Tris (hydroxymethyl) aminomethane (TRIZMA Base).

45. The medical dressing of claim 23, wherein the pharmaceutically acceptable pH buffering agent in solution adjusts the antimicrobial composition to a pH of about 7.0 to about 9.0.

46. The medical dressing of claim 44, wherein the pharmaceutically acceptable pH buffering agent in solution further adjusts the antimicrobial composition to a pH of about 8.0.

47. The medical dressing of claim 23, wherein the antimicrobial composition further comprises vitamin E.

48. A method for administering an antimicrobial agent to a human or animal having a skin injury or surface lesion, comprising the steps of:
a) identifying a skin injury or surface lesion of a human or an animal patient;
b) providing a medical dressing for delivering an antimicrobial composition to the skin injury or surface lesion, wherein the medical dressing comprises a support and an antimicrobial composition, wherein the antimicrobial composition comprises at least one pharmaceutically acceptable antimicrobial agent, at least one pharmaceutically acceptable chelating agent and at least one pharmaceutically acceptable pH buffering agent; and
c) applying the medical dressing to the skin injury or surface lesion of the human or animal patient thereby inhibiting the proliferation of a microbial infection thereof.

49. The method of claim 48, wherein the antimicrobial composition of step b) further comprises vitamin E.

50. A kit for preparing an antimicrobial composition for inhibiting the proliferation of a microbial infection or microbial colonization of a skin injury or surface lesion, comprising: packaging material comprising at least one vessel containing at least one pharmaceutically acceptable antimicrobial agent, at least one pharmaceutically acceptable chelating agent, at least one pharmaceutically acceptable pH buffering agent, and vitamin E, and instructions directing the use of the kit for preparing an antimicrobial composition for inhibiting the proliferation of a microbial population, and optionally for promoting tissue repair, of a skin injury or surface lesion of a human or animal.

51. The kit according to claim 50, further comprising instructions directing the use of the kit for applying the antimicrobial composition to a skin injury or surface lesion of a human or animal to inhibit the proliferation of a microbial infection thereof.

52. The kit as in claim 50, wherein the instructions direct the use of the antimicrobial composition for inhibiting an infection of a lesion of the oral mucosa of a human or animal patient.

53. The kit according to claim 50, further comprising a medical dressing, and instructions directing the use of the kit for preparing and applying the antimicrobial composition to the medical dressing and delivering the medical dressing to a human or animal to inhibit the proliferation of a microbial infection.
